(19) United States
(12) Patent Application Publication
(10) Pub. No.: US 2005/0283109 A1
(43) Pub. Date: Dec. 22, 2005

(54) Method and apparatus for lacrimal canal obstruction

(76) Inventor: Gholam A. Peyman, New Orleans, LA (US)

Correspondence Address: Bell, Boyd & Lloyd LLC, P.O. Box 1135, Chicago, IL, 60690-1135, US

(21) Appl. No.: 11/180,214

(22) Filed: Jul. 13, 2005

Related U.S. Application Data
(63) Continuation-in-part of application No. 11/039,696, filed on Jan. 18, 2005.
Application No. 11/039,696 is a continuation-in-part of application No. 10/625,543, filed on Jul. 24, 2003.
(60) Provisional application No. 60/452,554, filed on Mar. 7, 2003.

(51) Int. Cl. A61M 5/00

(52) U.S. Cl. 604/8

(57) ABSTRACT

A plug is presented. The plug includes biodendrimer and is suitably sized and shaped to be placed within a punctum or a lacrimal canal of an eye. The plug, when placed within the punctum or the lacrimal canal for approximately six months, can at least partly occlude the punctum or the lacrimal canal throughout the approximately six months. The plug can completely dissolve or partially dissolve and fall out of the punctum or the lacrimal canal after the approximately six months. A method of treating an eye is also presented. The method includes introducing biodendrimer into a punctum or a lacrimal canal of the eye and polymerizing the biodendrimer such that the polymerized biodendrimer at least partly occludes the punctum or the lacrimal canal for at least approximately six months.

### CROSS REFERENCE TO RELATED APPLICATIONS

This patent application is a continuation-in-part of U.S. Non-Provisional application Ser. No. 11/039,696, filed on Jan. 18, 2005, which is a continuation-in-part of U.S. Non-Provisional application Ser. No. 10/625,543, filed on Jul. 24, 2003, which claims the benefit of U.S. Provisional Application No. 60/452,554, filed on Mar. 7, 2003, the entire contents of all of which are incorporated herein by reference.

### BACKGROUND

One of the common complications of LASIK surgery is a condition known as “dry eye”. It is estimated that LASIK leads to the development of temporary dry eye in approximately 4% of patients because the nerves of the corneal flap are severed when forming the flap, leading to desensitization. Typically, the condition goes away approximately 6 months after the LASIK surgery, corresponding with the point in time at which the corneal flaps are re-innervated. However, the condition can lead to significant punctuate epithelial erosions and rose bengal staining on the flap, resulting in reduced vision quality or other visual disturbances.

One approach for treatment of temporary dry eye associated with LASIK is to occlude the punctum and/or lacrimal canal. Because tears drain away from the eye through the punctum, a small opening to the lacrimal canal of the eyelid, occluding the punctum and/or lacrimal canal can help in maintaining healthy eye moisture levels after LASIK surgery. Punctal or lacrimal occlusion can be achieved by cauterization, punctual/lacrimal plugs or punctual patches. Cauterization is traumatic and reversibility is not reliable. A punctual patch is formed by removing an area of epithelium and subcutaneous tissue from around the punctum (e.g., a 2 mm×2 mm area) and covering the punctum by replacing the removed tissue with a similar sized patch of bulbar conjunctiva or the inferior cul-de-sac. The patch is then sutured in place. However, punctual patches are also traumatic and a second procedure is required if the occlusion is only temporary (e.g., if occlusion is no longer needed once the corneal flap re-innervates).

There are two types of punctallacrimal plugs. One is a collagen plug that is designed to dissolve and/or be absorbed by the body in 4 to 7 days. This time period is insufficient for treating dry eye for the approximately 6 month period it takes for the corneal flap to re-innervate after LASIK surgery. Thus, these plugs are most often used as a diagnostic tool to determine whether more permanent plugs might be beneficial in solving the dry eye problem. The amount of time it takes for a plug to dissolve gives the doctor enough time to evaluate the problem, properly diagnose dry eye and determine whether a permanent punctal/lacrimal plug would be beneficial.

The other type of punctum/lacrimal plugs are silicone. Silicone punctal/lacrimal plugs are designed to be permanent and do not dissolve or absorb into the body. As a result, an additional procedure is necessary to remove the silicone punctal/lacrimal plugs once the corneal flap has re-innervated after LASIK surgery.

Thus, known punctal/lacrimal occlusion methods are traumatic, last insufficiently long to be an effective treatment for post-LASIK dry-eye treatment, and/or require subsequent procedures to reverse, sometimes with unreliable results.

### SUMMARY OF PREFERRED EMBODIMENTS

In one embodiment of the present invention, a plug is presented. The plug includes biodendrimer and is suitably sized and shaped to be placed within a punctum or a lacrimal canal of an eye. The plug, when placed within the punctum or the lacrimal canal for a time period, can at least partly occlude the punctum or the lacrimal canal throughout the time period. Further, the plug can completely occlude the punctum or the lacrimal canal throughout the time period. Also, the plug can completely dissolve or partially dissolve and fall out of the punctum or the lacrimal canal after the time period. The time period can be substantially more than seven days. Further, the time period can be two weeks, approximately six months or the amount of time it takes a corneal flap to re-innervate.

A method of treating an eye is also presented. The method includes introducing biodendrimer into a punctum or a lacrimal canal of the eye and polymerizing the biodendrimer such that the polymerized biodendrimer at least partly occludes the punctum or the lacrimal canal for a time period. Introducing biodendrimer into the punctum or the lacrimal canal can include injecting the biodendrimer with a syringe. Polymerizing the biodendrirner can include exposing the biodendrimer to laser light. Further, the laser light can be from an argon laser. The method can also include refraining from removing the polymerized biodendrimer from the punctum or the lacrimal canal before the polymerized biodendrimer completely dissolves or falls out of the punctum or the lacrimal canal. Also, the polymerized biodendrimer can completely dissolve or fall out of the punctum or the lacrimal canal after the time period. The time period can be substantially more than seven days. Further, the time period can be two weeks, approximately six months or the amount of time it takes a corneal flap to re-innervate.

Further, a method of retaining moisture in an eye is presented. The method includes making a biodendrimer plug. The biodendrimer plug is insertable into a punctum or a lacrimal canal of the eye such that the biodendrimer plug at least partly occludes the punctum or the lacrimal canal for a time period. Making the biodendrimer plug can include introducing biodendrimer into a mold. Also, making the biodendrimer plug can include exposing biodendrimer to laser light. Further, the laser light can be from an argon laser. The biodendrimer plug can completely dissolve or fall out of the punctum or the lacrimal canal if the biodendrimer plug is not removed from the punctum or the lacrimal canal. Further, the biodendrimer plug can completely dissolve or fall out of the punctum or the lacrimal canal after the time period. The time period can be substantially more than seven days. Further, the time period can be two weeks, approximately six months or the amount of time it takes a corneal flap to re-innervate.

Additional features and advantages of the present invention are described in, and will be apparent from, the following Detailed Description of Preferred Embodiments and the figures.

### BRIEF DESCRIPTION OF THE FIGURES

FIG. 1 is a front view of a portion of an eye showing a punctum without occlusion in accordance with one embodiment of the present invention.

FIG. 2 is a cross sectional view of the punctum of FIG. 1 taken along lines 2-2.

FIG. 3 is a front view showing a syringe with the tip of its needle in the punctum of FIG. 1.

FIG. 4 is a cross sectional view of the punctum and needle tip of FIG. 3 taken along lines 4-4.

FIG. 5 is a front view showing the punctum of FIG. 1 occluded.

FIG. 6 is a cross sectional view of the punctum and occlusion of FIG. 5 taken along lines 6-6.

FIG. 7 is a flow diagram of a process of occluding a punctum or lacrimal canal in accordance with one embodiment of the present invention.

FIG. 8 is a flow diagram of a process of occluding a punctum or lacrimal canal with a pre-formed plug in accordance with one embodiment of the present invention.

FIG. 9 is an elevational side view of a biodendrimer plug in accordance with one embodiment of the present invention.

FIG. 10 is an elevational side view of a biodendrimer plug that has ridges in accordance with one embodiment of the present invention.

FIG. 11 is a front view of the eye with the biodendrimer plug of FIG. 9 positioned within a punctum and lacrimal canal.

FIG. 12 is a front view of the eye with a biodendrimer plug completely embedded within the lacrimal canal.

FIG. 13 is an enlarged view of the lacrimal canal and biodendrimer plug of FIG. 12.

### DETAILED DESCRIPTION OF PREFERRED EMBODIMENTS

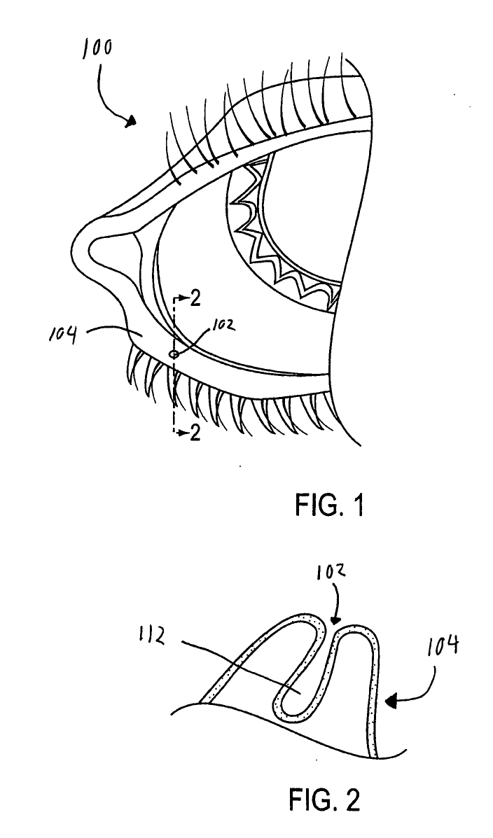

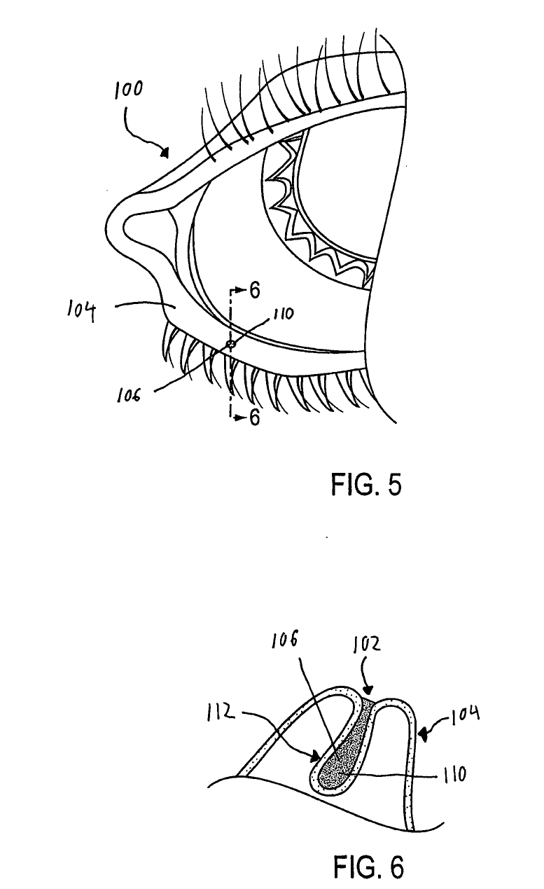

FIGS. 1-8 show a preferred process for occluding the punctum 102 and/or the lacrimal canal 112 of an eye 100. When the punctum 102 and lacrimal canal 112 are not occluded as shown in FIGS. 1-2, tears drain away from the eye 100 and into the nasal passages through the punctum 102 and lacrimal canal 112, located on the eyelid 104. It should be noted that both the upper and lower eyelids have punctums and lacrimal canals and that the techniques and devices described herein can be applied to either or both of the sets of punctums and lacrimal canals.

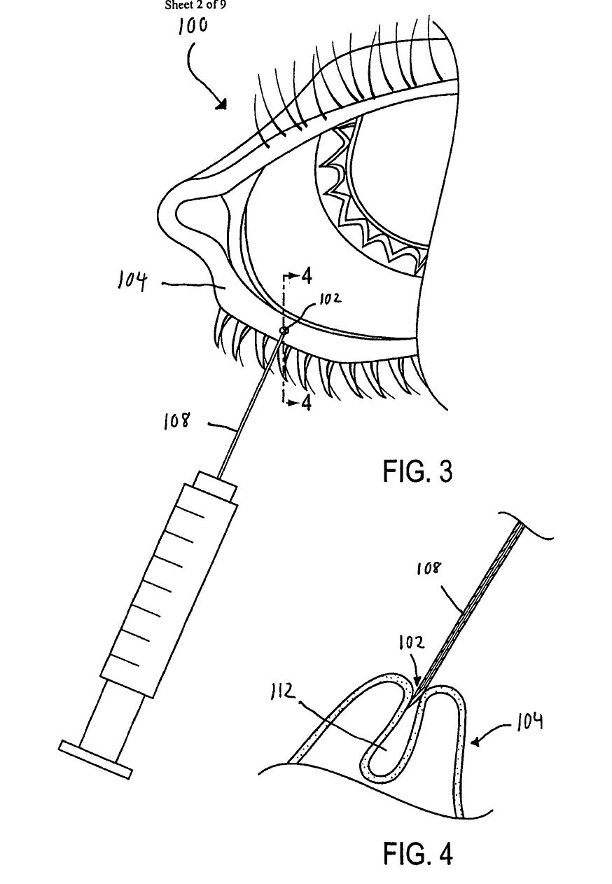

As shown in FIGS. 3-6, to at least temporarily occlude the punctum 102 and/or lacrimal canal 112, a bio-adhesive or any other suitable material, preferably biodendrimer 106, is introduced into the punctum 102 and/or the lacrimal canal 112. Biodendrimers include well-defined globular polymers containing a central core from which the polymer branches outward in a tree structure or fractal-like pattern. Biodendrimers can have properties including: (1) a single molecular weight (2) low viscosities, (3) high solubilities, and/or (4) a large number of end groups for functionalization. Further, biodendrimers are preferably composed of biocompatible monomers.

The biodendrimer 106 is preferably injected into the punctum 102 and/or lacrimal canal 112 using a syringe 108; however, the biodendrimer 106 can be introduced into the punctum 102 and/or lacrimal canal 112 using any suitable device.

Once the biodendrimer 106 is introduced into the punctum 102 and/or lacrimal canal 112, the biodendrimer 106 is preferably subjected to laser light to adhesively polymerize (i.e., harden) the biodendrimer 106 at a controlled rate such that the resulting biodendrimer punctal/lacrimal plug 110 continues to occlude the punctum 102 and/or lacrimal canal 112 for approximately six months before biodegrading/dissolving. However, the biodendrimer plug 110 can occlude the lacrimal canal 112 for substantially more than seven days, for approximately the time period it takes a corneal flap to re-innervate or any other suitable predetermined time period. Preferably, an argon laser supplies the laser light; however, any suitable laser light source can supply the laser light. Alternatively, the biodendrimer 106 can be polymerized by self-polymerization, chemical reaction, or any other suitable polymerization method.

After approximately 6 months, the corneal flap following LASIK surgery is re-innervated. As a result, the dry eye condition is substantially reduced or eliminated and a punctal/lacrimal plug is no longer necessary. Unlike punctal patches or silicone punctal/lacrimal plugs, there is no need for an additional procedure to remove a biodendrimer punctal/lacrimal plug 110.

Preferably, the biodendrimer punctal/lacrimal plug 110 completely dissolves and/or is absorbed by the body in approximately 6 months; however, the biodendrimer punctal/lacrimal plug can, in approximately 6 months, merely dissolve sufficiently that tears can once again pass through the punctum 102 and lacrimal canal 112 with the partly-dissolved biodendrimer punctal/acrimal plug 110 still partly occluding the punctum 102 and/or lacrimal canal 112 for an additional period of time, or the biodendrimer punctal/lacrimal plug 110 can dissolve sufficiently enough that the biodendrimer punctal/lacrimal plug 110 falls out of the punctum 102 and lacrimal canal 112 without the need of an extraction procedure, or the biodendrimer punctal/lacrimal plug 110 can dissolve to any other suitable degree. As a result, the punctum 102 and lacrimal canal 112 of the eye is once again not occluded as shown in FIGS. 1-2.

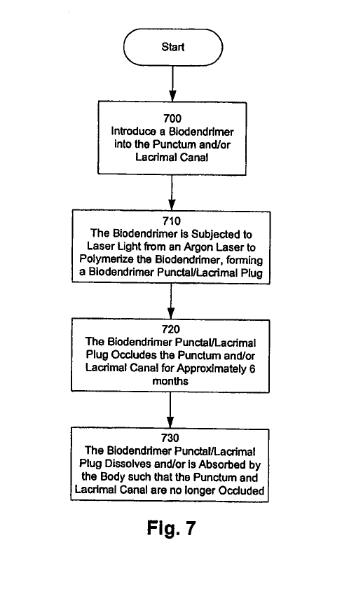

Thus, as shown in FIG. 7, a preferred procedure for temporarily occluding a punctum and/or lacrimal canal of an eye to treat dry eye following LASIK surgery includes introducing a biodendrimer into the punctum and/or lacrimal canal at step 700. Then, at step 710, the biodendrimer is subjected to laser light from an argon laser to polymerize the biodendrimer, forming a biodendrimer punctal/lacrimal plug. At step 720, the biodendrimer punctal/lacrimal plug occludes the punctum and/or lacrimal canal for approximately 6 months. Then, at step 730, the biodendrimer punctal/lacrimal plug dissolves and/or is absorbed by the body such that the punctum and lacrimal canal are no longer occluded and tears can once again drain through the punctum and lacrimal canal. As a result, tears are retained to combat dry eye while the corneal flap re-innervates and no extraction procedure is necessary to remove the punctal/lacrimal plug.

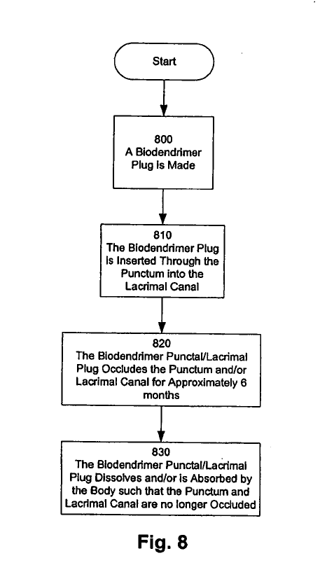

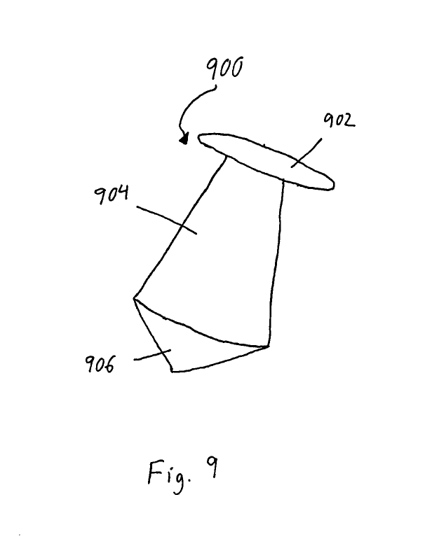

An alternative procedure for occluding the punctum and/or lacrimal canal of an eye to treat dry eye following LASIK surgery using a biodendrimer punctal/lacrimal plug is shown in FIG. 8. At step 800, a biodendrimer plug is made. Preferably, the biodendrimer plug is made by introducing biodendrimer into a mold and polymerizing the biodendrimer by subjecting it to laser light from an argon laser; however, the biodendrimer plug can be made using any suitable device and/or process. Further, the biodendrimer plug preferably has approximately the same size and/or shape as a silicone punctal/lacrimal plug; however the biodendrimer plug can have any suitable size and shape.

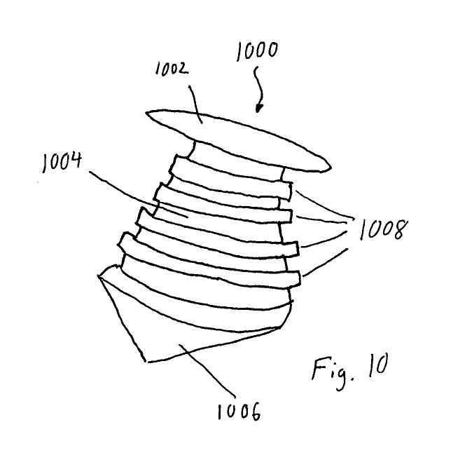

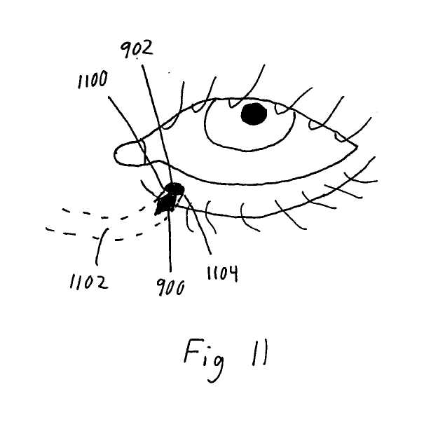

FIGS. 9-10 show preferred examples of pre-formed biodendrimer punctal/lacrimal plugs. The biodendrimer punctal/lacrimal plug 900 of FIG. 9 has a wide, substantially flat top 902, a substantially cylindrical main body 904 attached to the top 902, and a pointed section 906 attached to the main body 904 opposite the top 902. The main body 904 is preferably narrower near the top 902 than it is near the pointed section 906; however, the main body 904 can have any suitable configuration. The pointed section 906 is inserted first, thus facilitating the insertion of the main body 904 through the punctum and into the lacrimal canal. Preferably, the top 902 rests on the eyelid surface 1104, as shown in FIG. 11.

Similarly, the biodendrimer punctal/lacrimal plug 1000 of FIG. 10 has a wide, substantially flat top 1002, a substantially cylindrical main body 1004 attached to the top 1002, and a pointed section 1006 attached to the main body 1004 opposite the top 1002. The main body 1004 is narrower near the top 1002 than it is near the pointed section 1006; however, the main body 1004 can have any suitable configuration. The pointed section 1006 is inserted first, thus facilitating the insertion of the main body 1004 through the punctum and into the lacrimal canal. Preferably, the top 1002 rests on the eyelid surface. Additionally, the main body 1004 has ridges 1008. Preferably, the ridges 1008 aid in maintaining the position of the biodendrimer punctal/lacrimal plug 1000 upon insertion.

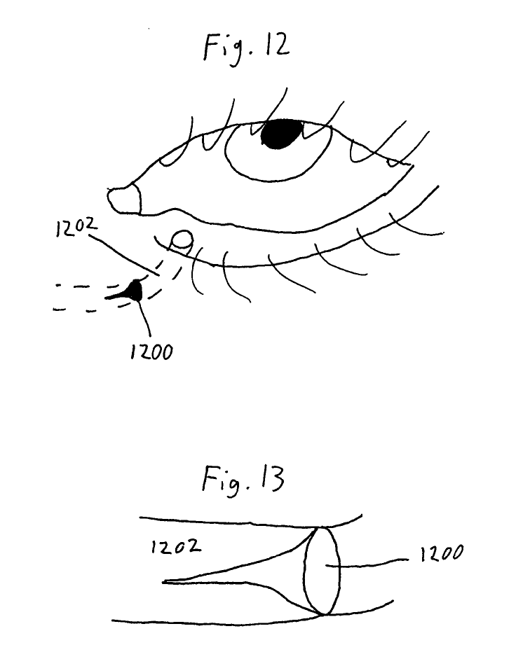

Returning to FIG. 8, after the biodendrimer punctal/lacrimal plug 900 is made, at step 810, the plug 900 is inserted through the punctum 1100 into the lacrimal canal 1102 as illustrated in FIG. 11. The top 902 preferably rests on the eyelid surface 1104. However, as illustrated in FIGS. 12 and 13, a biodendrimer punctal/lacrimal plug 1200 can be completely embedded in the lacrimal canal 1202. Further, it should be noted that the biodendrimer plug 1200 varies in structure from the plugs of FIGS. 9 and 10. The biodendrimer plug 1200 has a substantially flat top 1204 and a substantially cylindrical, pointed main body 1206; however, a biodendrimer plug can have any suitable configuration and is not required to have a substantially flat top or a substantially cylindrical main body.

As a result of the insertion of the biodendrimer punctal/lacrimal plug 900, at step 820, the biodendrimer punctal/lacrimal plug 900 occludes the punctum 1100 and/or lacrimal canal 1102 for approximately 6 months. Then, at step 930, the biodendrimer punctal/lacrimal plug 900 dissolves and/or is absorbed by the body such that the punctum 1100 and lacrimal canal 1102 are no longer occluded and tears can once again drain through the punctum 1100 and lacrimal canal 1102. As a result, tears are retained to combat dry eye while the corneal flap re-innervates and no extraction procedure is necessary to remove the punctal/lacrimal plug.

It should be noted that the techniques and devices described in this application are not limited to application to the punctum or the lacrimal canal or for treatment of dry eye due to desensitization of the corneal flap following LASIK surgery. Biodendrimer can be introduced, before or after polymerization, into any suitable duct, canal, or other suitable structure to occlude or partly occlude the structure temporarily without the need of an extraction procedure.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A plug comprising:
biodendrimer, wherein the plug is suitably sized and shaped to be placed within a punctum or a lacrimal canal of an eye.

2. The plug of claim 1, wherein the plug, when placed within the punctum or the lacrimal canal for about a predetermined time period, is configured to at least partly occlude the punctum or the lacrimal canal throughout the predetermined time period.

3. The plug of claim 2, wherein the plug is configured to completely occlude the punctum or the lacrimal canal throughout the predetermined time period.

4. The plug of claim 2, wherein the plug is configured to completely dissolve or partially dissolve and fall out of the punctum or the lacrimal canal after the predetermined time period.

5. The plug of claim 2, wherein the predetermined time period is substantially more than seven days.

6. The plug of claim 5, wherein the predetermined time period is about two weeks.

7. The plug of claim 5, wherein the predetermined time period is about six months.

8. The plug of claim 5, wherein the predetermined time period is approximately equal to the time period in which a corneal flap re-innervates.

9. A method of treating an eye comprising:
introducing biodendrimer into a punctum or a lacrimal canal of the eye; and
polymerizing the biodendrimer, wherein the polymerized biodendrimer at least partly occludes the punctum or the lacrimal canal for about a predetermined time period.

10. The method of claim 9, wherein introducing biodendrimer into the punctum or the lacrimal canal includes injecting the biodendrimer with a syringe.

11. The method of claim 9, wherein polymerizing the biodendrimer includes exposing the biodendrimer to laser light.

12. The method of claim 11, wherein the laser light is from an argon laser.

13. The method of claim 9, further comprising the step of:
refraining from removing the polymerized biodendrimer from the punctum or the lacrimal canal before the polymerized biodendrimer completely dissolves or falls out of the punctum or the lacrimal canal.

14. The method of claim 9, wherein the polymerized biodendrimer completely dissolves or falls out of the punctum or the lacrimal canal at about the predetermined time period.

15. The method of claim 14, wherein the predetermined time period is about six months.

16. A method of retaining moisture in an eye comprising:
making a biodendrimer plug, wherein the biodendrimer plug is insertable into a punctum or a lacrimal canal of the eye such that the biodendrimer plug at least partly occludes the punctum or the lacrimal canal for about a predetermined time period.

17. The method of claim 16, wherein making the biodendrimer plug includes introducing biodendrimer into a mold.

18. The method of claim 16, wherein making the biodendrimer plug includes exposing the biodendrimer to laser light.

19. The method of claim 16, wherein the biodendrimer plug completely dissolves or falls out of the punctum or the lacrimal canal if the biodendrimer plug is not removed from the punctum or the lacrimal canal.

20. The method of claim 16, wherein the biodendrimer plug completely dissolves or falls out of the punctum or the lacrimal canal after the predetermined time period.

21. The method of claim 16, wherein the predetermined time period is approximately six months.
